(19) United States
(12) Patent Application Publication
(10) Pub. No.: US 2020/0080120 A1
(43) Pub. Date: Mar. 12, 2020

(54) STEVIA SWEETENER AND MANUFACTURING PROCESS

(71) Applicant: QUFU SHENGREN PHARMACEUTICAL CO., LTD., Qufu (CN)

(72) Inventors: Laiwang ZHANG, Qufu (CN); Dongdong LIN, Qufu (CN); Yuqiang LAI, Qufu (CN)

(21) Appl. No.: 16/352,781

(22) Filed: Mar. 13, 2019

Related U.S. Application Data
(60) Provisional application No. 62/642,562, filed on Mar. 13, 2018.

(51) Int. Cl. C12P 19/18; C12P 19/56; A23L 27/30; A23L 2/60; A23P 10/40

(52) U.S. Cl. CPC C12P 19/18; C12P 19/56; C12Y 204/01019; A23L 2/60; A23P 10/40; A23L 27/36

(57) ABSTRACT

A method of preparing a glucosylated steviol glycoside composition involving by treating stevia materials with cyclodextrin glycosyltransferase.

### CROSS-REFERENCE TO RELATED APPLICATIONS

This application claims the benefit of priority under 35 U.S.C. §120 from U.S. Patent Application No. 62/642,562, filed on Mar. 13, 2018 and entitled STEVIA SWEETENER MANUFACTURING PROCESS, which is incorporated herein by reference in its entirety.

### BACKGROUND

Low-calorie drinks and foods have become increasingly popular for dietary purposes. Such low-calorie drinks and foods often contain sweeteners in place of sucrose. A popular class of sweeteners are the sweet-tasting glycosides of steviol, a diterpene derivative, extracted from the Stevia rebaudiana plant. The steviol molecule is glucosylated to form steviol glycosides such as stevioside, Rebaudioside A, Rebaudioside C, Dulcoside A, Rebaudioside D, Rebaudioside B, Rebaudioside F, Rebaudioside E, Steviolbioside, and Rubusoside, with the first 7 of these being relatively more abundant in extracts from the Stevia rebaudiana plant.

Steviol glycosides have a high level of sweetness. For example, stevioside has 250 to 300 times the sweetness of sucrose (Opinion on Stevioside as a Sweetener, CS/ADD/EDUL/167 final 17 Jun. 1999, Scientific Committee on Food, European Commission). However, steviol glycosides can have undesirable tastes or aftertastes in addition to sweetness, due at least in part to contaminants in the stevia extract. One way to improve the taste of steviol glycosides is to glycosylate semi-purified steviol glycosides with an enzyme. Methods of preparing compositions of glycosylated steviol glycosides have been disclosed for example in U.S. Pat. Nos. 8,993,269; 9,107,436; 9,474,296; 9,603,373; 9,615,599; and 9,706,792. Nevertheless; improved methods of preparing steviol glycoside compositions are still needed. The present invention is an improved method of preparing steviol glycoside compositions.

### SUMMARY

The present invention is directed to a method of preparing a steviol glycoside composition. In one embodiment, the method can comprise the following steps:
(a) providing a mixture of maltodextrin, stevioside, and water;
(b) adjusting the pH of the mixture to an acidic value;
(c) treating the acidic mixture of step (b) with cyclodextrin glycosyltransferase to obtain an enzyme-treated mixture;
(d) inactivating the cyclodextrin glycosyltransferase in the enzyme-treated mixture;
(e) adjusting the pH of the product of step (d) to 5.5-7.0;
(f) adding ethanol to the product of step (e);
(g) purifying the product of step (f) with resin;
(h) removing ethanol from the product of step (g) to form a sweetening mixture; and
(i) concentrating the sweetening mixture to form the steviol glycoside composition.

The method of preparing the steviol glycoside composition can also further comprise the following steps:
(j) converting the concentrated sweetening mixture in step (i) to a powder; and
(k) optionally packaging the powder.

### FIGURES

FIG. 1 is a flow chart showing one embodiment of the present method of preparing a steviol glycoside composition.

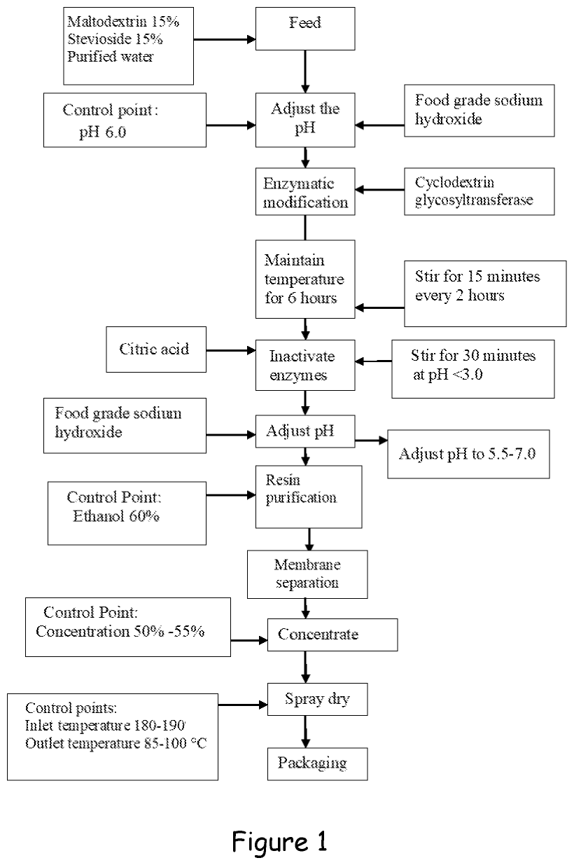

FIG. 2 is a flow chart showing an alternative embodiment of the present method of preparing a steviol glycoside composition.

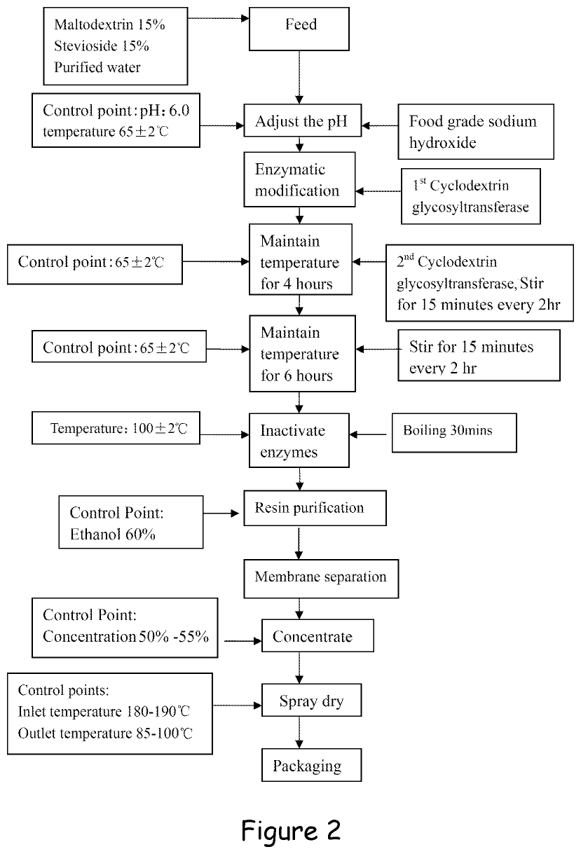

### DESCRIPTION

### Definitions

As used herein, the following terms and variations thereof have the meanings given below, unless a different meaning is clearly intended by the context in which such term is used.

“About” refers to an amount or quantity which is within 10% of the stated amount or quantity, either greater or less than the amount or quantity.

“Edible” refers to substances which can be ingested by a subject, preferably a mammalian subject, more preferably a human. “Edible” as used herein refers to both solid food and liquid beverages, as well as to medicinal products.

“Flavor” refers to the generally understood quality of substances perceived in the mouth and throat of a subject when substances are placed therein.

“Stevia” refers to the Stevia rebaudiana plant or to the leaves or other parts of this plant.

### Stevia Sweetener Manufacturing Process

The present method is a process for manufacturing a Stevia sweetener composition comprised of glycosylated steviol glycoside molecules. As used herein, the term “Stevia” refers to the Stevia rebaudiana plant or to leaves or other parts of this plant, or to compositions derived from the Stevia rebaudiana plant.

In one embodiment, the present process begins with soaking Stevia leaves in water, which can be at room temperature, and then mixing the leaves with a two-dimensional mixer. This extracts stevioside and other steviol glycosides into the water. After this, a flocculent (a substance that promotes the clumping of suspended particles) is added to the Stevia leaf suspension, and this mixture is then passed through a screen filter in order to remove the flocculent and other solid materials. The screened liquid is purified to separate steviol glycosides from other components of the aqueous mixture. In one embodiment, the aqueous mixture can be passed over an ion exchange resin, such as an absorption and/or adsorption column, which retains stevioside and other steviol glycosides. Activated carbon can be used as the ion exchange resin, for example, as it assists in decolorizing the retained material. Salts and other colored components of the screened liquid pass through the column and are discarded. Steviol glycosides can also be concentrated and separated from other components of the aqueous mixture through the use of a membrane separation process known to the art, which can be used instead of or in addition to purification by an ion exchange resin.

When an ion exchange resin is used to purify extracted Stevia materials, the steviol glycosides adsorbed to the resin can be released with ethanol or other solvents and/or solutions known to the art. For example, a solution of food grade alcohol (ethanol) can then passed through the resin in order to release the steviol glycosides from the column. This alcohol mixture can then also be passed through a polymer membrane.

The stevioside mixture is then evaporated, such as by placing it under reduced pressure in order to evaporate water and remaining alcohol from the mixture. Preferably, the evaporated mixture is spray dried when it reaches an appropriate concentration, such as through high-speed centrifugal spray drying, in order to make solid stevioside powders from the liquid stevioside mixture. Alternatively, steviol glycosides can be separated from other components of the liquid stevioside mixture by crystallizing them from the mixture, such as through ethanol recrystallization.

The steviol glycoside mixture comprising stevioside and other steviol glycosides which was obtained by the foregoing steps can then be used for the further manufacture of the present composition comprising glycosylated steviol glycosides, or alternatively steviol glycosides derived in a different manner can be used. The steviol glycosides are added to water to form a “feed” mixture preferably comprising an amount of about 15 wt % of stevioside and/or other steviol glycosides, optionally with other ingredients. In some embodiments, maltodextrin is added, such as in amounts of between 5 wt % and 20 wt %, preferably in amounts of about 10 wt % or about 15 wt % of maltodextrin, and water is provided (with “wt %” meaning weight % based on the total weight of the mixture). The pH is adjusted, for example to between about 6.0 and 7.0, preferably to about 6.0, which can be accomplished with food grade sodium hydroxide or other additives known to the art to form an acidic mixture. The acidic mixture is preferably maintained at a temperature of between 60° C. and 80° C., such as at about 60° C., about 65° C., or about 70° C.

The acidic mixture is then subjected to a first enzymatic modification by treating it with cyclodextrin glycosyltransferase to obtain a first enzyme-treated mixture. Cyclodextrin glycosyltransferase can be added to the acidic mixture in amounts known to the art, such as in amounts in which the ratio of a cyclodextrin glycosyltransferase to the steviol glycosides 1:1 or less on a molar basis, such as from about 0.15 to about 0.99, from about 0.25 to about 0.95, from about 0.3 to about 0.9, from about 0.35 to about 0.6, or from about 0.4 to about 0.75. The amount of cyclodextrin glycosyltransferase for example can be between about 1 wt % and about 10 wt %, between about 3 wt % and about 8 wt %, for example 4 wt %, 5 wt %, or 6 wt %. The first enzymatic modification can be conducted at a temperature of between about 60° C. and about 80° C., such as at about 60° C., about 65° C., or about 70° C. The mixture is preferably reacted for between about 4 hours and about 10 hours, such as for about 6 hours. The mixture is preferably stirred for about 15 minutes every 2 hours.

In some embodiments, following the first cycle of enzymatic modification, a further amount of cyclodextrin glycosyltransferase is added to the first enzyme-treated mixture in an amount as discussed above for the first enzymatic modification, thereby forming a second enzyme-treated mixture. The second enzymatic modification can be conducted at a temperature of between about 60° C. and about 80° C., such as at about 60° C., about 65° C., or about 70° C. The mixture is preferably reacted for between about 4 hours and about 10 hours, such as for about 6 hours. The mixture is preferably stirred for about 15 minutes every 2 hours. The use of a second enzyme modification cycle results in a final product which is more completely glucosylated.

Following enzymatic modification, the cyclodextrin glycosyltransferase in the product mixture is inactivated. In one embodiment, this is accomplished by exposing the enzyme to citric acid, preferably at a pH of less than 3.0. Optionally, after the addition of citric acid, the mixture is stirred for 30 minutes. The pH of the inactivated enzyme solution is then adjusted with the addition of food grade sodium hydroxide, such as to a pH of between about 5.0 and about 7.0, such as to a pH of about 5.5-6.5.

In an alternative embodiment, the cyclodextrin glycosyltransferase in the product mixture is inactivated by raising the temperature of the second enzyme-treated mixture to greater than 90° C., preferably to about 100° C. The second enzyme-treated mixture is maintained at this temperature for between about 15 minutes and about 1 hour, preferably for between about 20 minutes and about 45 minutes, such for about 30 minutes or about 40 minutes. Lower lengths of time are preferred in order to avoid degrading the glucosylated steviol glycosides of the composition. Enzyme inactivation at elevated temperatures reduces the risk of introducing impurities into the product, and helps to avoid degradation of the final product.

The resulting solution is then preferably purified, such as by passing the solution through a macroporous adsorption column. For example, a polypropylene adsorption column using a macroporous resin made of styrene and divinylbenzene copolymer can be used. Ethanol at a concentration of 60% by volume is then added to the macroporous membrane to release the absorbed steviol glycoside material. The ethanol is then preferably removed with a polymer membrane, separating the ethanol from the resulting purified glucosylated steviol glycoside composition.

The purified glucosylated steviol glycoside composition is then preferably concentrated to between about 50 wt % and about 55 wt %. Optionally, this step can comprise concentrating the purified glucosylated steviol glycoside composition by evaporation at about 95±2° C. to reach a concentration of 50 wt %-55 wt %, for example. The purified glucosylated steviol glycoside composition can then be made into a powder, such as through spray drying. Optionally, spray drying can be accomplished with a spray dryer having an inlet temperature of about 180-190° C. and an outlet temperature of about 85° C. to about 100° C. The powder obtained in this step is then preferably sieved with a magnetic vibration sieve having a pore size of less than 180 microns before packaging. Optionally, the sieved powder can be blended homogeneously with a mixture to obtain the present glucosylated steviol glycoside composition.

### Stevia Sweetener Composition

The foregoing process results in a flavor modifying composition which generally imparts sweetness to foods, beverages, medicinal products, and other compositions. The composition preferably comprises greater than 65% glucosylated steviol glycosides, 20%-30% residual steviol glycosides, less than 5.5% rebaudioside A, and less than 1% maltodextrin. In a preferred embodiment, the composition comprises at least 67% glucosylated steviol glycosides, between about 18% and about 28% residual steviol glycosides, less than about 4.5% rebaudioside A, and less than 0.5% maltodextrin. The total amount of steviol glycosides of the composition is preferably more than about 95%.

The foregoing glucosylated steviol glycoside composition can be used as a flavor modifying composition in order to sweeten, mask, and/or otherwise modify the flavor of foods, beverages, medicaments, and/or other edible compositions. The glucosylated steviol glycoside composition is a flavor modifying composition that can be added directly to an edible composition in order to modify its flavor. The glucosylated steviol glycoside composition can also be combined with fillers, other sweeteners, and/or additional additives known for use with sweeteners in order to create a flavor modifying composition. For example, maltodextrin can be included in the formulation of the sweetener.

### EXAMPLES

### Example 1

A glucosylated steviol glycoside composition was prepared by following the sequential steps in Table 1 below.

TABLE 1

| Step No. | Process Step | Manufacturing Action | Materials & Conditions |
|---|---|---|---|
| 1. | Thermostat | Water was pumped into a | The temperature was raised to 50° C. |
| | | dissolution tank | |
| 2. | Feed | The raw material sugar and | Raw sugar: 15% |
| | | auxiliary maltodextrin were added | Maltodextrin: 15% |
| | | into the reaction tank with solvent | Solvent: 70% |
| | | (water) | |
| 3. | Adjust the pH | Food grade sodium hydroxide used | pH: 6.0 |
| | | to adjust pH | |
| 4. | Enzymatic | Cyclodextrin glycosyltransferase | Enzyme amount: 4% by weight |
| | modification | added | |
| 5. | Stir | Stirred liquid thoroughly | Stir for 30 minutes |
| 6. | Warming | Warmed the liquid after adding the | Temperature: 80 ± 2° C. |
| | | enzyme. | Holding time: 6 hours; stir 15 |
| | | | minutes every 2 hours |
| 7. | Inactivate | Added citric acid to kill enzyme | pH <3.0 stirred for 30 minutes |
| | enzymes | | |
| 8. | Adjust pH | Added food grade sodium | pH 5.5~7.0 |
| | | hydroxide | |
| 9. | Resin purification | Added the reacted sugar solution | Macroporous adsorption column |
| | | into a macroporous adsorption | rinsed with purified water to |
| | | column | colorless; adsorption amount |
| | | | 9T/45 min. |
| | | | Analyze the amount with 60% ± 2% |
| | | | ethanol. Analytical amount |
| | | | 9T/min |
| 10. | Remove alcohol | Used membrane separation | Pass the analytic solution through a |
| | | | polymer membrane to separate |
| | | | ethanol from stevioside |
| | | | composition; the stevioside |
| | | | composition is concentrated below |
| | | | until alcohol is removed |
| 11. | Concentrate | Feed solution was concentrated by | Concentrating temperature: 95° C. ± 2 |
| | | evaporation | Concentrated liquid sugar content of |
| | | | 50-55% |
| 12. | CCP2 Spray | The concentrated feed liquid was | Entering air temperature: 180~200° C. |
| | drying | sprayed into a spray dryer by a | Exiting air temperature: 90~100° C. |
| | | pipe to be dried to form a solid | |
| | | powder | |
| 13. | Magnetic | After spray-drying, powder placed | Magnetic force ≥10000 gauss |
| | Sieve | over magnetic vibration sieve | Material loading speed 6 kg/min |
| | | (Magnetic oscillation screen) | |
| 14. | Mixing | The material was mixed | Mixing time: 500 kg mixed for 1 |
| | | homogeneously by mixer | hour |

The product resulting from the foregoing process was analyzed and found to have the chemical formula C₃₈H₆₀O₁₈ and a particle size of 80 mesh. This composition includes greater than 65% glucosylated steviol glycosides, 20%-30% residual steviol glycosides, less than 5.5% rebaudioside A, and less than 1% maltodextrin.

The composition further had the properties shown in Table 2 below.

TABLE 2
Properties of Composition

| Item | Testing Method | Specification |
|---|---|---|
| Appearance | Visual | powder |
| Color | Visual | white |
| pH Value | 1 in 100 | 4.5-7.0 |
| Total Glucoside Content % | HPLC | ≥95 |
| Reacted steviol glycosides | HPLC | ≥65 |
| content % | | |
| Sweetness | GB8270 | ≥260 |
| Ash % | Muffle Furnaces | ≤0.1 |
| Moisture % | Moisture Balance | ≤6.0 |
| (Pb) Heavy Metal(Pb) ppm | AAS | ≤1 ppm |
| (As) Arsenic ppm | AAS | ≤0.1 ppm |
| Methanol ppm | GC | ≤200 |
| Ethanol ppm | GC | ≤5000 |
| Microbiological Analysis: | | |
| Total Plate Count | <2021> | <1000 cfu/g |
| Yeast/Mold | <2021> | <100 cfu/g |
| Staphylococcus aureus: | <2022> | Negative |
| E. Coli: | <2022> | Negative |
| Salmonella SPP | AOAC/<2022> | Negative |

### Example 2

A glucosylated steviol glycoside composition was prepared by following the sequential steps in Table 2 below.

TABLE 2

| Step No. | Process Step | Manufacturing Action | Materials & Conditions |
|---|---|---|---|
| 1. | Thermostat | Water was pumped into a | The temperature was raised to 50° C. |
| | | dissolution tank | |
| 2. | Feed | The raw material sugar and | Raw sugar: 15% |
| | | auxiliary maltodextrin were added | Maltodextrin: 15% |
| | | into the reaction tank with solvent | Solvent: 70% |
| | | (water) | |
| 3. | Adjust the pH | Food grade sodium hydroxide used | pH: 6.0 |
| | | to adjust pH | Temperature: 65 ± 2° C. |
| 4. | Enzymatic | Cyclodextrin glycosyltransferase | Enzyme amount: 4% by weight |
| | modification | added | Temperature: 65 ± 2° C. |
| 5. | Stir | Stirred liquid thoroughly | Stir for 30 minutes |
| 6. | First enzymatic | Maintained temperature for 4 | Temperature: 65 ± 2° C. |
| | reaction | hours. | Holding time: 4 hours; stir 15 |
| | | | minutes every 2 hours |
| 7. | Second enzymatic | Maintained temperature for 6 hours | Temperature: 65 ± 2° C. |
| | reaction | | Holding time: 6 hours; stir 15 |
| | | | minutes every 2 hours |
| 8. | Inactivate | Boiling for 30 minutes | Temperature: 100 ± 2° C. |
| | enzymes | | |
| 9. | Resin purification | Added the reacted sugar solution | Macroporous adsorption column |
| | | into a macroporous adsorption | rinsed with purified water to |
| | | column | colorless; adsorption amount |
| | | | 9T/45 min. |
| | | | Analyze the amount with 60% ± 2% |
| | | | ethanol. Analytical amount |
| | | | 9T/min |
| 10. | Remove alcohol | Used membrane separation | Pass the analytic solution through a |
| | | | polymer membrane to separate |
| | | | ethanol from stevioside |
| | | | composition; the stevioside |
| | | | composition is concentrated below |
| | | | until alcohol is removed |
| 11. | Concentrate | Feed solution was concentrated by | Concentrating temperature: 95° C. ± 2 |
| | | evaporation | Concentrated liquid sugar content of |
| | | | 50-55% |
| 12. | CCP2 Spray | The concentrated feed liquid was | Entering air temperature: 180~200° C. |
| | drying | sprayed into a spray dryer by a | Exiting air temperature: 90~100° C. |
| | | pipe to be dried to form a solid | |
| | | powder | |
| 13. | Magnetic | After spray-drying, powder placed | Magnetic force ≥10000 gauss |
| | Sieve | over magnetic vibration sieve | Material loading speed 6 kg/min |
| | | (Magnetic oscillation screen) | |
| 14. | Mixing | The material was mixed | Mixing time: 500 kg mixed for 1 |
| | | homogeneously by mixer | hour |

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure.

The term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise. Ranges which are described as being “between” two values include the indicated values, as the recitation of value ranges herein is merely intended to serve as a shorthand method for referring individually to each separate value falling within the range. Unless otherwise indicated herein, each individual value is incorporated into the specification as if it were individually recited herein. All references cited herein are incorporated by reference in their entirety.

What is claimed is:

1. A process for producing a highly purified glucosyl Stevia composition, comprising the steps of:
(a) providing steviol glycosides extracted from Stevia rebaudiana in an acidic solution, thereby forming an acidic mixture;
(b) contacting the acidic mixture with a first amount of cyclodextrin glycosyltransferase to form a first enzyme treated mixture;
(c) maintaining the first enzyme treated mixture at a temperature of about 65° C. for between 4 and 10 hours;
(d) contacting the first enzyme treated mixture with a second amount of cyclodextrin glycosyltransferase to form a second enzyme treated mixture;
(e) maintaining the second enzyme treated mixture at a temperature of about 65° C. for between 6 and 10 hours;
(f) inactivating the cyclodextrin glycosyltransferase in the second enzyme treated mixture by raising the temperature of the second enzyme treated mixture to between about 90° C. and about 100° C., thereby forming an enzyme inactivated mixture; and
(g) purifying the enzyme inactivated mixture to form the glucosyl Stevia composition.

2. The process of claim 1, wherein prior to step (a) the process comprises the steps of:
(i) soaking Stevia rebaudiana materials in water to extract steviol glycosides, thereby forming an aqueous mixture;
(ii) passing the aqueous mixture over a column containing activated charcoal, thereby adsorbing steviol glycosides on the activated charcoal; and
(iii) eluting the adsorbed steviol glycosides from the activated charcoal.

3. The process of claim 1, wherein the acidic mixture of step (a) is at a temperature of about 65° C.

4. The process of claim 1, wherein the acidic mixture comprises about 15 weight percent steviol glycosides.

5. The process of claim 1, wherein the acidic mixture comprises about 15 weight percent maltodextrin.

6. The process of claim 1, wherein the first enzyme treated mixture at a temperature of about 65° C. for about 4 hours.

7. The process of claim 1, wherein the second enzyme treated mixture is maintained at a temperature of about 65 ° C. for between 6 hours.

8. The process of claim 1, wherein the cyclodextrin glycosyltransferase in the second enzyme treated mixture is inactivated by raising the temperature of the second enzyme treated mixture to about 100° C.

9. The process of claim 1, wherein the glucosyl Stevia composition is spray dried to form a powder.

10. A flavor modifying composition derived from Stevia rebaudiana, comprising at least 65% glucosylated steviol glycosides, between about 20% and about 30% residual steviol glycosides, and less than about 4.5% rebaudioside A, wherein the amount of glucosylated steviol glycosides and residual steviol glycosides is greater than 95%.

11. The flavor modifying composition of claim 10, wherein the composition comprises at least 67% glucosylated steviol glycosides.

12. The flavor modifying composition of claim 10, wherein the composition comprises between about 18% and about 28% residual steviol glycosides.

13. The flavor modifying composition of claim 10, wherein the composition comprises less than about 0.5% maltodextrin.

14. The flavor modifying composition of claim 10, wherein the composition has a particle size of 80 mesh.

15. A method of altering the flavor of an edible product, comprising adding the flavor modifying composition of claim 10 to the edible product.

16. The method of claim 15, comprising the step of adding the flavor modifying composition to a food product.

17. The method of claim 15, comprising the step of adding the flavor modifying composition to a beverage.

18. The method of claim 15, comprising the step of adding the flavor modifying composition to a medicinal product.
